(19) United States
(12) Patent Application Publication
(10) Pub. No.: US 2008/0132766 A1
(43) Pub. Date: Jun. 5, 2008

(54) Surgical Access System And Method Of Using Same

(75) Inventors: Jack A. Dant, St. Paul, MN (US); Hugh D. Hestad, Edina, MN (US)

Correspondence Address: WOOD, HERRON & EVANS (ZIMMER SPINE), 2700 CAREW TOWER, 441 VINE STREET, CINCINNATI, OH, 45202, US

(73) Assignee: ZIMMER SPINE, INC., Minneapolis, MN (US)

(21) Appl. No.: 11/566,913

(22) Filed: Dec. 5, 2006

(51) Int. Cl. A61B 1/32

(52) U.S. Cl. 600/219

(57) ABSTRACT

A system for creating an access portal to a surgical site generally comprises a first portal member configured to be inserted through an incision and toward the surgical site and a second portal member configured to be inserted through the incision for engagement with proximate the first portal member. Each portal member includes a body with opposed side edges. The side edges of the first portal member cooperate with the side edges of the second portal member to secure the access system within the incision with the bodies of the respective portal members defining the portal to the surgical site.

### FIELD OF THE INVENTION

The present invention relates generally to access systems useful in various surgical procedures, and more particularly to an access system useful for minimally invasive surgical procedures.

### BACKGROUND OF THE INVENTION

To perform a surgical procedure at a location on a patient's body, a surgeon typically makes an incision at the location and retracts surrounding tissue to provide access to a surgical site. More specifically, retractors are used to pull tissue away from the incision and maintain access to the surgical site throughout the procedure. Occasionally one or more surgical assistants are present during the procedure to manually hold the retractors in position. The presence of assistants, however, can crowd the operating area and leave the surgeon with less space to move about the surgical site and complete the surgical procedure. Moreover, assistants are not always readily available or do not have sufficient time to devote to holding a retractor for extended periods of time when other patients need immediate care.

As a result, in many instances a surgeon places some sort of frame or anchoring device near the surgical site. A wide variety of anchoring devices exist in the marketplace, each incorporating clamps, notches, adjustable arms, or other mechanical devices for securing retractors during a surgical procedure. The anchoring devices, however, present the same challenges associated with assistants manually holding the retractors—they occupy space around the incision and may interfere with the surgeon's movements.

Additionally, for many surgical procedures, simply making an incision and retracting tissue may not provide sufficient access to the surgical site without significant drawbacks. This is especially true when the surgical site is located deep within a patient's body. For example, in the past, surgical procedures for anterior and posterior spinal surgery required relatively large incisions to effectively operate on the spinal elements. Relatively large incisions are generally undesirable because they may result in increased damage to muscle tissue, increased blood loss, prolonged pain to the patient, and potential scarring.

To minimize these undesirable aspects, many surgical procedures are now conducted using minimally invasive techniques. These techniques involve creating a relatively small incision and then increasing the effective size of the incision opening using various dilators. Dilation, in effect, splits the muscle tissue as opposed to cutting the muscle tissue, which in turn causes less damage to the muscle, increases recovery times, and reduces patient discomfort. Retractors are used after or during dilation to hold open the incision and passageway through the soft tissue.

One method of dilating tissue involves making a small incision and inserting a guidewire through the incision to the surgical site. A first dilator with a central channel or bore is placed over the guidewire and advanced toward the surgical site. Successively larger dilators are then advanced, one at a time, over the first dilator to expand the opening of the incision. After the largest dilator has been inserted through the incision, the smaller dilators and guidewire may be removed from the patient's body. Alternatively, each dilator may be removed from the body as soon as a larger dilator is advanced over it. As the incision is dilated and the largest dilator establishes a path to the surgical site, a retractor or cannula is inserted through or over the largest dilator. The cannula provides the necessary retraction when the largest dilator is removed so as to establish an unencumbered path, or working channel, to the surgical site. A surgeon may use the working channel to visualize the surgical site and insert tools to complete the particular surgical procedure.

Although such minimally invasive techniques may reduce the undesirable aspects associated with large incisions, the dilation procedure can be time-consuming and labor intensive. Several dilators may be required to increase the opening of the incision to an effective size for performing a surgical procedure. Each dilator must be carefully inserted through the incision to prevent traumatic displacement of muscle tissue. On the other hand, if a relative small number of dilators are used, the cannula inserted through the incision is typically limited in size. A smaller working channel makes it more difficult to visualize the surgical site and manipulate tools to complete the surgical procedure.

Some manufacturers have attempted to address these challenges by providing expandable retractors. For example, U.S. Patent Application Serial No. 2006/0004401 discloses an elongated retractor and dilator for accessing a surgical site along the spine. In the '041 application, the retractor includes a frame having first and second frame portions coupled to a pair of generally straight, parallel arms or rails. An elongated body segment or blade extends from each of the frame portions so as to be substantially perpendicular to the arms. The body segments are inserted through an incision following a dilation procedure to provide a generally straight walled access path to the surgical site. The frame portions are then moved with respect to each other along the arms to separate the body portions and form an elliptically-shaped working channel that increases the effective size of the incision opening.

Although expandable retractors such as that described above generally improve access to the surgical site, there remains room to improve such products. For example, movement of the body segments away from each other typically creates a gap or spacing between the longitudinal or side edges of the body segments on opposed sides of the working channel. Additionally, many expandable retractors include a number of parts which makes the operation of such devices more complex. Locking mechanisms or external forces must typically be provided to maintain the retractors in an expanded configuration because of the forces imposed by the surrounding tissue.

Thus, as can be appreciated, there is a need for an improved system for establishing access to a surgical site. The system should minimize the undesirable effects associated with relatively large surgical incisions, yet address the challenges associated with current retractors used in minimally invasive procedures. As such, the system should have a simple design and be easy to operate, without the need for a labor-intensive dilation procedure involving many components or an expandable retractor.

### SUMMARY OF THE INVENTION

The present invention provides a system and method for creating an access portal to a surgical site. The system generally comprises a first portal member configured to be inserted through an incision and toward the surgical site. A second portal member is configured to be inserted through the incision for engagement with the first portal member. The first and second portal members cooperate to retract tissue from the incision and to define the access portal to the surgical site.

In one embodiment of the invention, the first and second portal members each have a body with a concave portion and opposed side edges. After inserting the first portal member through an incision, the second portal member may be inserted through the incision in a nested configuration with the first portal member. In such a configuration the opposed side edges of the second portal member are offset from the opposed side edges of the first portal member, which allows the concave portions of the first and second portal members to be nested with respect to each other. This arrangement reduces the amount of muscle tissue that must be split in order for the incision to accommodate the second portal member.

Once inserted into the incision, the first and second portal members may then be repositioned to a portal configuration in which the side edges of the second portal member engage the side edges of the first portal member. Repositioning therefore involves moving the concave portions out of the nested configuration to retract tissue and expand a space between the first and second portal members. Respective handle members may be coupled to the body of each portal member to facilitate this repositioning. In the portal configuration, the concave portions are aligned with each other to define an access portal or space to the surgical site. Because the forces exerted by the surrounding muscle tissue on the portal members keep the side edges engaged with each other, the handle members may be subsequently removed from the bodies, if desired, prior to completing a surgical procedure through the access portal.

In a further aspect or embodiment of the invention, the second portal member may be aligned with the first portal member prior to being inserted through the incision. Such an embodiment may eliminate the need to reposition the second portal member after it is inserted through the incision. For example, the body of the second portal member may be sized to be received between the opposed side edges of the first portal member. Thus, after inserting the first portal member through the incision, the second portal member may be aligned between the side edges of the first portal member and inserted through the incision at an angle. Inserting the second portal member in this manner gradually splits muscle tissue to expand the space between the portal members. The second portal member eventually straightens out as it approaches the surgical site so that the concave portions of the first and second portal members define an access portal to the surgical site.

### BRIEF DESCRIPTION OF THE DRAWINGS

The accompanying drawings, which are incorporated in and constitute a part of this specification, illustrate exemplary embodiments of the invention and, together with a general description of the invention given above, and the detailed description given below, serve to explain the principles of the invention.

FIG. 1 is a perspective view of a surgical access system according to one embodiment of the invention;

FIG. 2 is a perspective view showing the access system of FIG. 1 inserted through an incision to establish a portal to a surgical site within a body;

FIGS. 3A-3C are top elevational views sequentially illustrating a method of using the access system of FIG. 1 to establish a portal within a body;

FIGS. 4 and 5 are perspective views illustrating a surgical access system according to another embodiment of the invention being inserted into an incision to establish a portal; and

FIG. 6 is a top elevational view showing the surgical access system of FIGS. 4 and 5 defining a portal to a surgical site.

### DETAILED DESCRIPTION OF THE PREFERRED EMBODIMENTS

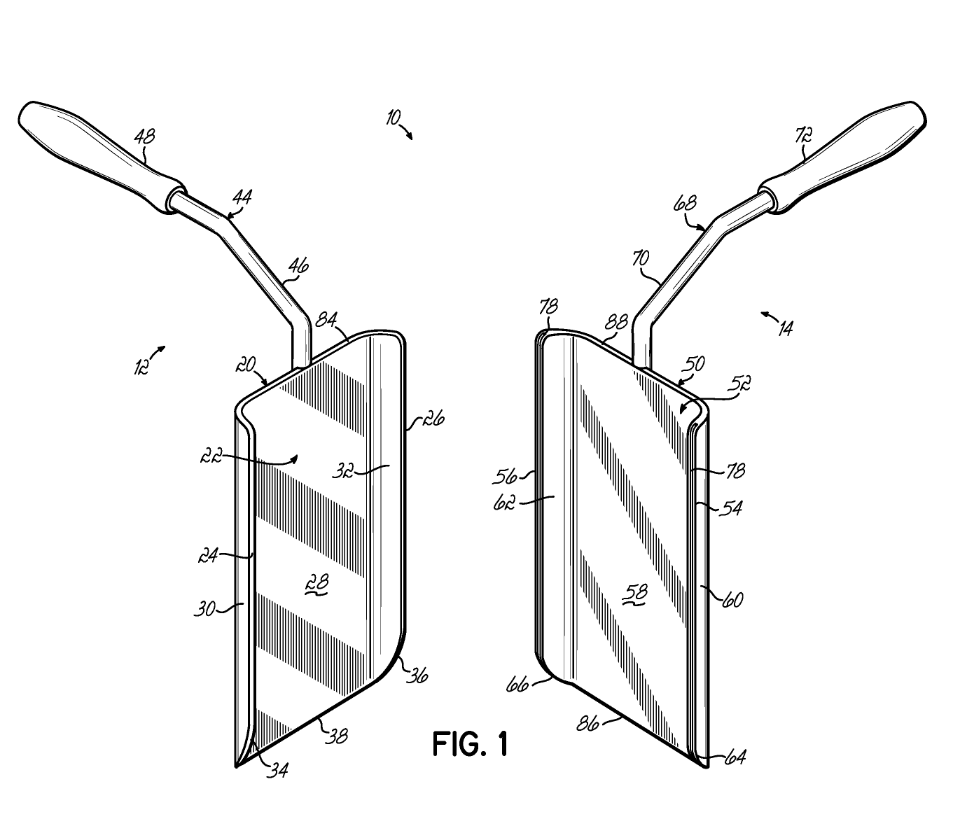

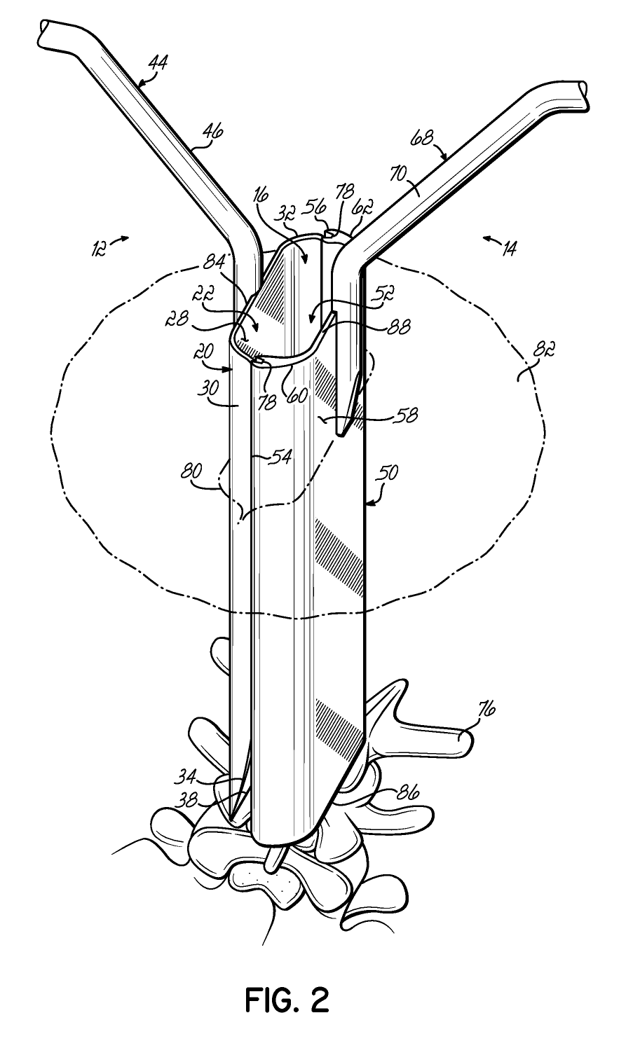

With reference to FIG. 1, a surgical access system 10 is shown according to one embodiment of the invention. The access system 10 generally comprises a first portal member 12 and second portal member 14. The first and second portal members 12, 14 cooperate with each other to define an access portal 16 (FIG. 2) to a surgical site within a body, as will be described in greater detail below. Although only two portal members are shown, those skilled in the art will appreciate that the system 1 0 may include more than two portal members. For example, the system 10 may include three or four portal members (not shown) configured to define an access portal when properly arranged. The shape and the size of the portal members 12, 14 may therefore be varied to accommodate the design of the system 10 and to operate in accordance with the principles discussed below.

As shown in FIG. 1, the first portal member 12 includes an elongate body 20 having a concave portion 22 and opposed side edges 24, 26. The concave portion 22 may have a circular, rectangular, arcuate, or any other cross-sectional configuration adapted to define a portion of an access portal 16 (FIG. 2). Thus, the term concave simply refers to any non-planar configuration facing inward such that an indentation or pocket is formed to define a portion of the portal 16. In the exemplary embodiment shown in FIG. 1, the concave portion 22 is coextensive with the body 20 and defined by a substantially flat wall 28 with opposed side walls 30, 32 extending upwardly from the wall 28. The side walls 30, 32 terminate at the opposed side edges 24, 26 and may be curved with respect to the flat wall 28 so as to provide the body 20 with a substantially C-shaped or arcuate profile. Respective tapered portions 34, 36 are provided on each side wall 30, 32 to provide a transition from a bottom edge 38 of wall 28 to the side edges 24, 26. Although the first portal member 12 is shown as having a symmetrical configuration, the side walls 30, 32 may be designed with different sizes, profiles, curvatures, etc. Similarly, although the wall 28 is shown as substantially planar, the wall 28 may alternatively be designed with one or more curved portions (not shown).

A handle member 44 may be coupled to the body 20 to facilitate manipulation of the first portal member 12. The handle member 44 includes a shaft 46 extending away from the body 20 at an angle and a grip portion 48 coupled to the shaft 46. If desired, the shaft 46 may be selectively coupled to the body 20 so that the handle member 44 is removable. For example, the shaft 46 may be configured to engage a slot (not shown) provided on a back surface of the wall 28 to couple the handle member 44 to the body 20. After the first portal member 12 is positioned in an incision and manipulated in the manner discussed below, the shaft 46 may be removed from the body 20 so that the surgeon can easily access the portal 16 (FIG. 2). A wide variety of removable handle members exist in the surgical tool market. Thus, it will be appreciated that the first portal member 12 may incorporate a wide variety of handle types and mechanisms for selectively coupling the handle member 44 to the body 20.

The second portal member 14 has substantially the same configuration as the first portal member 12. In other words, the second portal member 14 comprises a body 50 having a concave portion 52 with opposed side edges 54, 56. The concave portion 52 is defined by a substantially flat wall 58 with opposed side walls 60, 62 curving upwardly from the wall 58. Respective tapered portions 64, 66 are provided on the opposed side walls 60, 62 to facilitate insertion through an incision, and a handle member 68 includes a shaft 70 coupled to the body 50 and a grip portion 72 to facilitate manipulation. As with the first portal member 12, a wide variety of alternative shapes and configurations are possible for the body 50 and handle member 68. The second portal member 14 therefore need not have substantially the same configuration as the first portal member 12. The first and second portal members 12, 14 may also be constructed from the same or different materials. In one embodiment, the bodies 20, 50 of the first and second portal members 12, 14 are constructed from a radiolucent material.

As shown in FIGS. 1 and 2, the opposed side edges 54, 56 of second portal member 14 are configured to cooperate with the opposed side edges 24, 26 of first portal member 12 to secure the first and second portal members 12, 14 within an incision 80 with the concave portions 22, 52 defining the access portal 16 to a surgical site. More specifically, in the embodiment shown in FIGS. 1 and 2, the opposed side edges 54, 56 of second portal member 14 each define a channel or groove 78 adapted to receive one of the opposed side edges 24, 26 of first portal member 12. The side edges 24, 26 are maintained in the channels 78 by the forces exerted on the first and second portal members 12, 14 by the surrounding muscle tissue. The muscle tissue tends to resist any separation or displacement, such as that caused by the insertion of the first and second portal members 12, 14 and formation of the portal 16. Although the surgical site is shown as being spinal elements 76 (FIG. 2), the access system 10 may be used to establish a portal 16 to surgical sites elsewhere on a body.

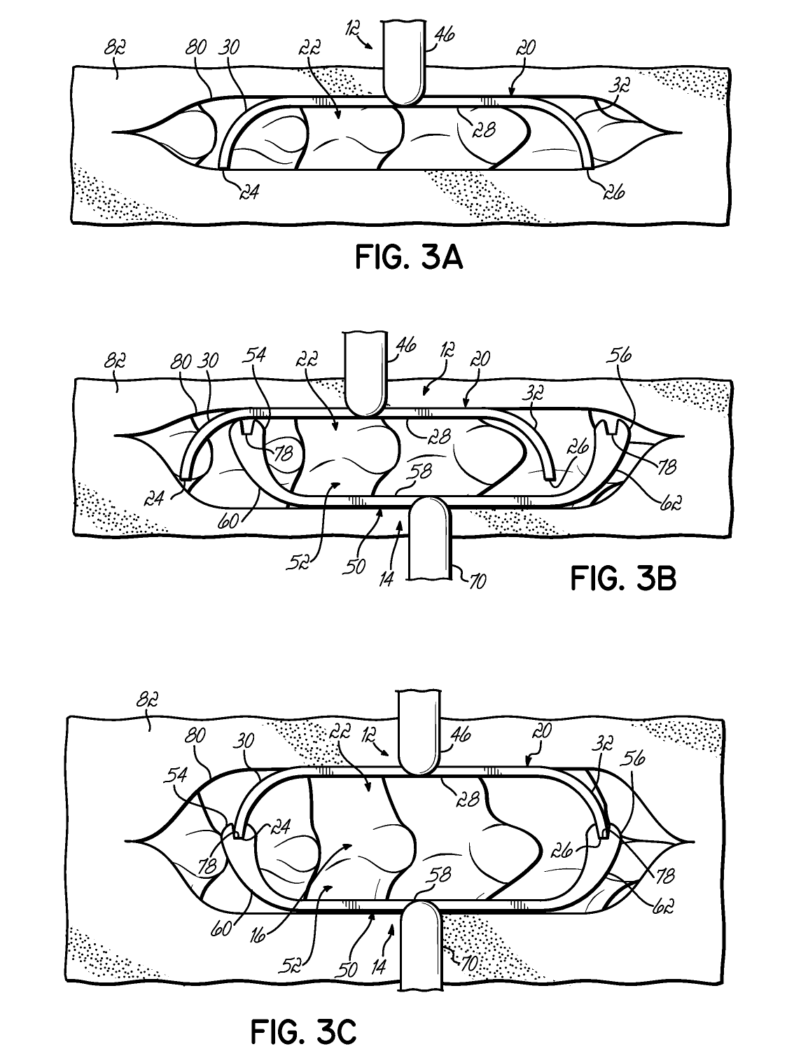

A method of using the surgical access system 10 will now be described. As shown in FIG. 3A, a relatively small incision 80 is made on a body 82 to begin a minimally invasive procedure. The first portal member 12 is then inserted through the incision 80 and toward a surgical site. As the tapered portions 34, 36 of first portal member 12 pass through the incision 80 and split the surrounding muscle tissue, the incision 80 expands to accommodate the body 20 of first portal member 12. Eventually the first portal member 12 is received in the body 82 with the bottom edge 38 positioned proximate the surgical site. A top edge 84 (FIG. 2) of first portal member 12 and the handle member 44 may remain outside the body 82. If desired, a lip or flange (not shown) may be provided on the body 20 of first portal member 12 proximate the top edge 84 to further retract tissue away from the incision 80.

Once the first portal member 12 is positioned within the incision 80, the second portal member 14 may be inserted through the incision 80 proximate the first portal member 12 as shown in FIG. 3B. For example, the second portal member 14 may be inserted through the incision 80 with the concave portion 52 nested with the concave portion 22 of first portal member 12. The opposed side edges 54, 56 of second portal member 14 are offset from the opposed side edges 24, 26 of first portal member 12 in this nested configuration. Such an arrangement minimizes the space between the first and second portal members 12, 14. As a result, the incision 80 does not need to significantly expand to accommodate the second portal member 14.

The second portal member 14 is inserted into the body 82 until a bottom edge 86 (FIG. 2) is generally positioned proximate the surgical site. As with the first portal member 12, the second portal member 14 may further include a lip or flange (not shown) extending outwardly from a top edge 88 to further retract tissue from the incision 80. When inserted, the tissue around the first and second portal members 12, 14 resists further expansion. In other words, the tissue forces the first and second portal members 12, 14 toward each other so as to maintain the nested configuration shown in FIG. 3B.

To expand the space between the first and second portal members 12, 14, an individual manipulates at least one of the first and second portal members 12, 14 using the associated handle member. For example, an individual may hold the first portal member 12 steady by gripping the handle member 44 (FIG. 2) with one of his or her hands and then manipulate the second portal member 14 by moving the handle member 68 with his or her other hand. Alternatively, the individual may move both the first and second portal members 12, 14 by simultaneously moving the handle members 44, 68 with his or her hands. Sufficient force is applied during this manipulation to overcome the resistance of the muscle tissue surrounding the first and second portal members 12, 14.

As shown in FIG. 3C, eventually the first and second portal members 12, 14 are aligned so that the side edges 24, 26 engage the side edges 54, 56. It will be appreciated that the first and second portal members 12, 14 may be designed to provide any type of locking arrangement between the side edges 24, 26, 54, 56. In the exemplary embodiment shown in FIGS. 1-3C, the side edges 24, 26 of first portal member 12 are received in the channels or grooves 78 of second portal member 14. Such an arrangement prevents the first and second portal members 12, 14 from moving laterally with respect to each other. Additionally, the force of the surrounding tissue retains the first and second portal members 12, 14 in this portal configuration with the concave portions 22, 52 defining an access portal 16 to the surgical site.

The access portal or space 16 provides an unobstructed path to the surgical site. A surgeon may use the portal 16 to visualize the surgical site or to insert tools and complete a surgical procedure. To further facilitate access to the surgical site, the handle members 44, 68 may be removed from the first and second portal members 12, 14. Such a step simply involves uncoupling the shafts 46, 70 from the respective bodies 20, 50. Removing the handle members 44, 68 allows a surgeon to move easily about the portal 16 to complete the particular surgical procedure. Alternatively, the handle members 44, 68 may be manipulated after the portal 16 is established to increase or decrease the exposure at the surgical site. For example, the handle members 44, 68 may be manipulated to shift or direct the portal 16 from a first region of the surgical site to a second region of the surgical site.

The system 10 therefore provides a simple and easy method for establishing an access portal 16 to a surgical site. Due to the simple design of the system 10, the method does not require making a relatively large incision into the body 82. Instead, the small incision 80 is made and slightly expanded by inserting the first and second portal members 12, 14 and splitting muscle tissue. This avoids the need for a dilation process involving numerous components. If desired, however, conventional dilation procedures could still be used to expand the incision 80 prior to establishing the portal 16. Additionally, although the access system 10 addresses some of the challenges associated with expandable retractors, such devices may still be used in combination with the access system 10 if desired.

After completing a surgical procedure, the first and second portal members 12, 14 may be removed from the body 82 by reversing the steps above. Thus, the shafts 46, 70 are coupled to the respective bodies 20, 50 if the handle members 44, 68 were previously removed. The first and second portal members 12, 14 may then be manipulated using the handle members 44, 68 to release the side edges 24, 26 from the side edges 54, 56. This allows the first and second portal members 12, 14 to be removed from the body 82, one at a time, using the handle members 44, 68. Alternatively, the side edges 24, 26 may remain engaged with the side edges 54, 56 while the first and second portal members 12, 14 are removed from the body 82 simultaneously.

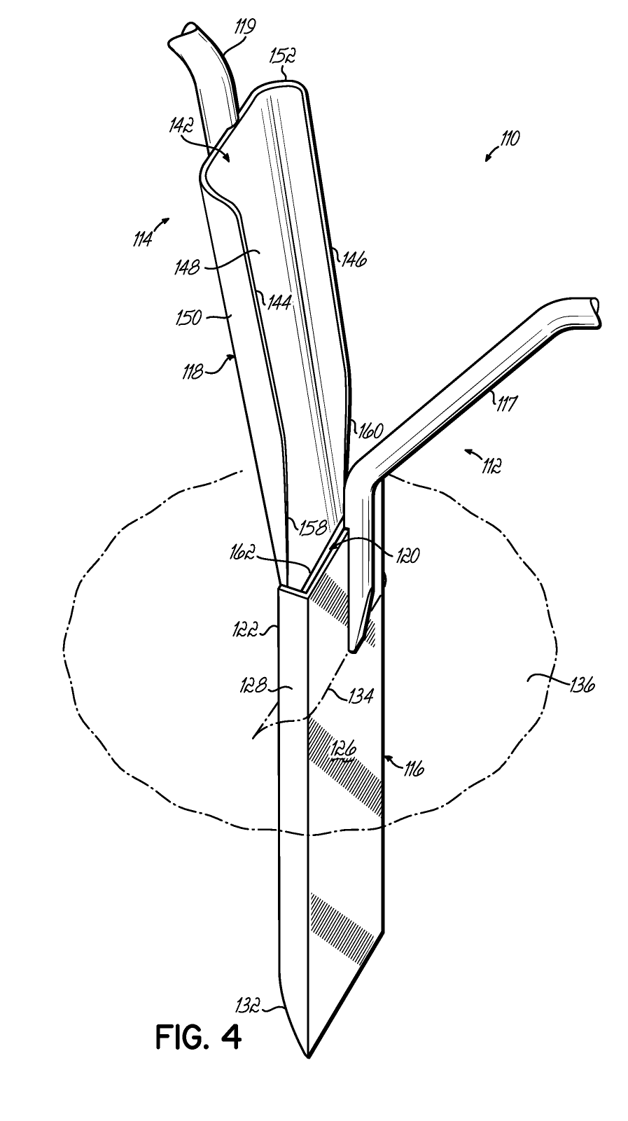

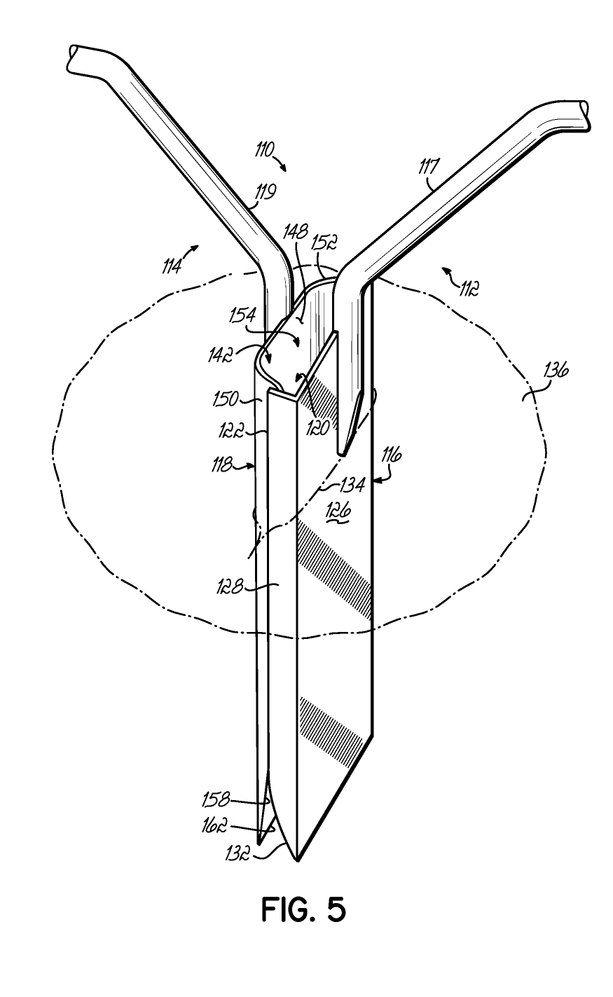

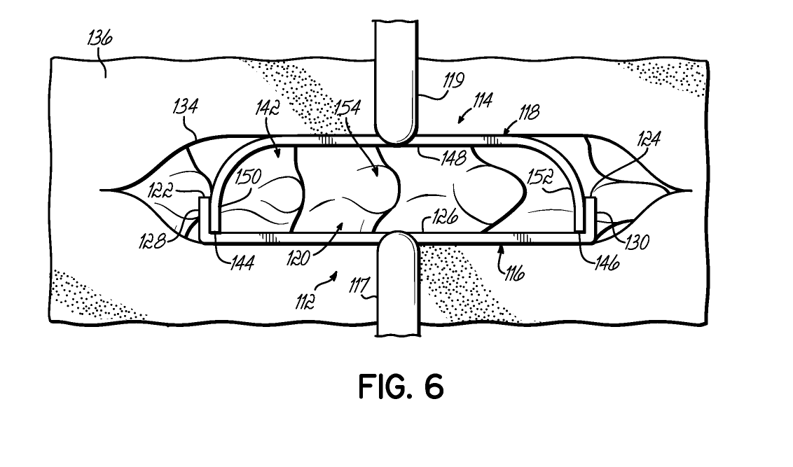

FIGS. 4-6 illustrate an access system 110 according to another embodiment of the invention. The access system 110 also includes first and second portal members 112, 114 having respective bodies 116, 118 and handle members 117, 119. The handle members 117, 119 are only partially shown in FIGS. 4-6 because they operate upon the same principles that were discussed with respect to the handle members 44, 68. Accordingly, reference can be made to the above description of the handle members 44, 68 and only the differences between the system 10 and the system 110 will be described below.

The body 116 of first portal member 112 includes a concave portion 120 with opposed side edges 122, 124. Once again, the term concave simply refers to any non-planar configuration facing inward such that an indentation or pocket is formed between the opposed side edges 122, 124. The concave portion 120 is defined by a substantially flat wall 126 and opposed side walls 128, 130 extending upwardly from the substantially flat wall 126. In the embodiment shown in FIGS. 4-6, the opposed side walls 128, 130 are substantially perpendicular to the flat wall 126. A tapered portion 132 may be provided on each of the side walls 128, 130 to facilitate insertion through an incision 134 and into a body 136.

The second portal member 114 is sized to be received between the opposed side edges 122, 124 of first portal member 112. More specifically, the body 118 of second portal member 114 includes a concave portion 142 and opposed side edges 144, 146. The concave portion 142 is defined by a substantially flat wall 148 and opposed side walls 150, 152 curving upwardly from the substantially flat wall 148. The opposed side walls 150, 152 are positioned proximate to the opposed side walls 128, 130 of first portal member 112 when the body 118 is aligned with the first portal member 112. Such an arrangement prevents the first and second portal members 112, 114 from shifting laterally with respect to each other and defines an access portal 154 due to the interaction between the concave portions 120, 142.

Thus, in use, the first portal member 112 is inserted through the incision 134 and toward a surgical site. The body 118 of second portal member 114 is then aligned between the opposed side edges 122, 124 of the first portal member 112 as shown in FIG. 4. Because the opposed side walls 150, 152 include relative long tapered portions 158, 160 to provide a gradual transition from a bottom edge 162 of body 118, the second portal member 114 may be inserted at an angle with respect to the first portal member 112. Inserting the second portal member 114 in this manner gradually expands the incision 134 as the bottom edge 162 approaches the surgical site.

Eventually the second portal member 114 straightens out with the opposed side edges 144, 146 resting against the substantially flat wall 126 of first portal member 112. By this point, the incision 134 has been expanded and the concave portion 142 of second portal member 114 cooperates with the concave portion 120 of first portal member 112 to define the access portal or space 154. Therefore, unlike the access system 10, the system 110 does not require manipulating the first and second portal members 112, 114 from a nested configuration to a portal configuration after they have been inserted through the incision 134. The portal 154 is instead gradually established as the second portal member 114 is inserted through the incision 134 proximate the first portal member 112.

While the invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. For example, the second portal member 14 may be inserted into the body 82 in the portal configuration with the concave portions 22, 52 defining the access portal 16. Such a method may not involve a nested configuration. Additionally, the first and second portal members 12, 14 or 112, 114 may be inserted into the body simultaneously. The invention in its broader aspects is therefore not limited to the specific details, representative apparatus and methods and illustrative examples shown and described. Accordingly, departures may be made from such details without departing from the scope or spirit of Applicants' general inventive concept.

What is claimed is:

1. A system for creating an access portal from an incision to a: surgical site, comprising:
a first portal member configured to be inserted through the incision and toward the surgical site, the first portal member having a body with a concave portion and opposed side edges; and
a second portal member configured to be inserted through the incision for engagement with the first portal member, the second portal member having a body with a concave portion and opposed side edges, the opposed side edges of the second portal member being configured to cooperate with the opposed side edges of the first portal member to secure the first and second portal members within the incision with the concave portions defining the access portal to the surgical site.

2. The system of claim 1, wherein the concave portion of the first portal member is defined by a substantially flat wall and opposed side walls extending upwardly from the substantially flat wall.

3. The system of claim 2, wherein the opposed side walls curve upwardly from the substantially flat wall.

4. The system of claim 2, wherein the opposed side walls each include a tapered portion configured to facilitate inserting the first portal member through the incision and toward the surgical site.

5. The system of claim 2, wherein the concave portion of the first portal member is coextensive with the body of the first portal member.

6. The system of claim 1, wherein the first portal member further includes a handle member coupled to the body.

7. The system of claim 6, wherein the handle member is selectively coupled to the body of the first portal member so that the handle member may be removed when the first portal member is secured to the second portal member.

8. The system of claim 6, wherein the handle member further comprises a shaft extending away from the body of the first portal member at an angle and a grip portion coupled to the shaft.

9. The system of claim 1, wherein at least one of the first and second portal members further includes a flange extending from the body and away from the concave portion, the flange being configured to retract tissue away from the incision.

10. The system of claim 1, wherein the bodies of the respective first and second portal members are formed from a radiolucent material.

11. A method of creating an access portal from an incision to a surgical site, comprising:
making an incision on a body;
inserting a first portal member through the incision and toward the surgical site, the first portal member having a body with a concave portion and opposed side edges; and
inserting a second portal member through the incision for engagement with the first portal member, the second portal member having a body with a concave portion and opposed side edges, the opposed side edges of the second portal member being configured to cooperate with the opposed side edges of the first portal member to secure the first and second portal members within the incision with the concave portions defining the access portal to the surgical site.

12. The method of claim 11, wherein inserting the second portal member further comprises aligning the body of the second portal member between the opposed side edges of the first portal member.

13. The method of claim 12, the body of the second portal member having a bottom edge, the concave portion of the second portal member including a tapered portion to provide a transition from the bottom edge to the opposed side edges, and wherein inserting the second portal member further comprises aligning the tapered portion with a surface on the first portal member so that the second portal member is inserted through the incision at an angle.

14. The method of claim 11, wherein inserting the first portal member further comprises manipulating a handle member coupled to the body of the first portal member.

15. The method of claim 14, further comprising:
uncoupling the handle member from the first portal member.

16. The method of claim 11, further comprising:
inserting a surgical instrument through the access portal to perform a surgical operation at the surgical site.

17. A method of creating an access portal from an incision to a surgical site, comprising:
making an incision on a body;
inserting a first portal member through the incision and toward the surgical site, the first portal member having a concave portion with opposed side edges;
inserting a second portal member through the incision with a concave portion of the second portal member nested with the concave portion of the first portal member, the second portal member having opposed side edges offset from the opposed side edges of the first portal member in the nested configuration;
repositioning at least one of the first and second portal members to retract tissue from the incision and expand a space between the first and second portal members; and
engaging the side edges of the first portal member with the side edges of the second portal member to secure the first and second portal members within the incision with the concave portions defining the access portal to the surgical site.

18. The method of claim 17, further comprising:
inserting a surgical instrument through the access portal to perform a surgical operation at the surgical site.

19. The method of claim 17, further comprising:
removing the first and second portal members simultaneously from the incision.

20. The method of claim 17, further comprising:
releasing the side edges of the first portal member from the side edges of the second portal member;
repositioning the first and second portal members to a nested configuration to decrease the space between the first and second portal members;
removing the first portal member from the incision; and
removing the second portal member from the incision.

21. The method of claim 17, wherein repositioning at least one of the first and second portal members further comprises moving a handle member coupled the corresponding portal member.

22. The method of claim 21, further comprising:
uncoupling the handle member from the corresponding portal member.

23. The method of claim 17, wherein engaging the side edges of the first portal member with the side edges of the second portal member further comprises positioning the side edges of the first portal member in respective channels defined by the side edges of the second portal member.

24. A system for creating an access portal from an incision to a surgical site, comprising:
a first portal member configured to be inserted through the incision and toward the surgical site, the first portal member having a body with a concave portion and opposed side edges; and
a second portal member having a body with a concave portion and opposed side edges, the second portal member configured to be inserted through the incision with the concave portion of the second portal member nested with the concave portion of the first portal member, the side edges of the second portal member being offset from the side edges of the first portal member in the nested configuration;
wherein at least one of the first and second portal members are moveable to a portal configuration in which the side edges of the first portal member engage the side edges of the second portal member to secure the first and second portal members within the incision with the concave portions defining the access portal to the surgical site.

25. The system of claim 24, wherein at least one of the opposed side edges of the second portal member define a channel adapted to receive one of the opposed side edges of the first portal member.

26. A system for creating an access portal from an incision to a surgical site, comprising:
a first portal member configured to be inserted through the incision and toward the surgical site, the first portal member having a body with a concave portion and opposed side edges; and
a second portal member configured to be inserted through the incision for engagement with the first portal member and between the opposed side edges;
wherein the first and second portal members are configured to define the access portal to the surgical site.

27. A method of creating an access portal from an incision to a surgical site, comprising:
making an incision on a body;
inserting a first portal member through the incision and toward the surgical site, the first portal member having a body with a concave portion and opposed side edges;
aligning a body of a second portal member between the opposed side edges of the first portal member; and
inserting the second portal member through the incision proximate the first portal member.
